(19) United States
(12) Patent Application Publication
(10) Pub. No.: US 2021/0182936 A1
(43) Pub. Date: Jun. 17, 2021

(54) SYSTEM AND METHOD FOR PRODUCT SELECTION

(71) Applicant: KONINKLIJKE PHILIPS N.V., EINDHOVEN (NL)

(72) Inventors: Praveen Kumar Pandian Shanmuganathan, Monroeville, PA (US); Jonathan Sayer Grashow, Monroeville, PA (US)

(21) Appl. No.: 17/110,359

(22) Filed: Dec. 3, 2020

Related U.S. Application Data
(60) Provisional application No. 62/946,995, filed on Dec. 12, 2019.

(51) Int. Cl. G06Q 30/06; G06Q 10/08; G16H 40/20; G06F 16/22; A61M 16/06

(52) U.S. Cl. CPC G06Q 30/0631; G06Q 10/087; A61M 16/0605; G06F 16/22; G16H 40/20

(57) ABSTRACT

A system including a user information database structured to store first information corresponding to a consumer and a first fit score corresponding to a current product associated with the consumer, a product information database structured to store second information corresponding to a new product, a communication unit structured to communicate with a user device, and a processing unit including a fit score determination module structured to determine a second fit score of the new product based on the first information and the second information, to determine if the second fit score is greater than the first fit score, and to control the communication unit to push a notification to the user device in response to determining that the second fit score is greater than the first fit score.

### CROSS-REFERENCE TO RELATED APPLICATIONS

This patent application claims the priority benefit under 35 U.S.C. § 119(e) of U.S. Provisional Application No. 62/946,995, filed on Dec. 12, 2019, the contents of which are herein incorporated by reference.

### BACKGROUND OF THE INVENTION

### 1. Field of the Invention

The disclosed concept relates to devices for assisting a user with selecting a product such as a patient interface device. The disclosed concept also relates to methods for assisting a user with selecting a product such as a patient interface device.

### 2. Description of the Related Art

Obstructive sleep apnea (OSA) is a condition that affects millions of people from around the world. OSA is characterized by disturbances or cessation in breathing during sleep. OSA episodes result from partial or complete blockage of airflow during sleep that lasts at least 10 seconds and often as long as 1 to 2 minutes. In a given night, people with moderate to severe apnea may experience complete or partial breathing disruptions as high as 200-500 per night. Because their sleep is constantly disrupted, they are deprived of the restorative sleep necessary for efficient functioning of body and mind. This sleep disorder has also been linked with hypertension, depression, stroke, cardiac arrhythmias, myocardial infarction and other cardiovascular disorders. OSA also causes excessive tiredness.

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because patient interface devices are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. Additionally, an improperly fitted mask can cause red marks or pressure sores on the face of the patient. Another concern is that an improperly fitted patient interface device can include gaps between the patient interface device and the patient that cause unwanted leakage and compromise the seal between the patient interface device and the patient. A properly fitted patient interface device should form a robust seal with the patient that does not break when the patient changes positions or when the patient interface device is subjected to external forces. Thus, it is desirable to properly fit the patient interface device to the patient.

3D scanning can be employed in order to improve the fit of the patient interface device to the patient. Generally, a 3D scan can be taken of the patient's face and then the information about the patient's face can be used to select the best fitting patient interface device, to customize an existing patient interface device, or to custom make a patient interface device that fits the patient well.

After initially selecting a patient interface device, a patient will generally continue using that patient interface device. A patient will generally only replace the patient interface if there is a problem with it or it becomes worn out.

### SUMMARY OF THE INVENTION

Accordingly, it is an object of the disclosed concept to provide targeted notifications to consumers that a more suitable product is available. It is also an object of the disclosed concept to periodically or, in response to new product availability, analyze the suitability of products for consumers and notify the consumer if a more suitable product is available.

As one aspect of the disclosed concept, a system comprises: a user information database structured to store first information corresponding to a consumer and a first fit score corresponding to a current product associated with the consumer; a product database structured to store second information corresponding to a new product; a communication unit structured to communicate with a user device; and a processing unit including a fit score determination module structured to determine a second fit score of the new product based on the first information and the second information, to determine if the second fit score is greater than the first fit score, and to control the communication unit to push a notification to the user device in response to determining that the second fit score is greater than the first fit score.

As one aspect of the disclosed concept, a method of assisting with product selection comprises: obtaining first information corresponding to a consumer and a first fit score corresponding to a current product associated with the consumer; obtaining second information corresponding to a new product; determining a second fit score of the new product based on the first information and the second information; determining if the second fit score is greater than the first fit score; and in response to determining that the second fit score is greater than the first fit score, pushing a notification to a user device.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a product selection system in accordance with an example embodiment of the disclosed concept;

FIG. 2 is a schematic diagram of a server for product selection in accordance with an example embodiment of the disclosed concept;

FIG. 3 is a flowchart of a method of assisting with product selection in accordance with an example embodiment of the disclosed concept; and

FIG. 4 is a flowchart of a method of assisting with product selection in accordance with another example embodiment of the disclosed concept.

### DETAILED DESCRIPTION OF EXEMPLARY EMBODIMENTS

As required, detailed embodiments of the disclosed concept are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the disclosed concept in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the context of patient interface devices, a patient interface device is recommended to the patient based on various characteristics of the patient. The suitability of a patient interface device for a patient is referred to as a fit score. A patient interface with a high fit score will be recommended to the patient over a patient interface device with a low fit score. The fit score of a patient interface device may be determined based on the various characteristics of the patient. For example, the geometry of the patient's face may be one such characteristic. The geometries of the patient's face and the patient interface may be compared to determine how well the patient interface device will fit the patient. A better fitting patient interface device will may have a higher fit score while a worse fitting patient interface may have a lower fit score. Other characteristics may also be taken into account, such as the condition the patient seeks to treat using the patient interface device. Some types of patient interface devices may not be suitable for treating a particular condition, which could lower the fit score of those patient interface devices. Other characteristics, such as the patient's preferences or the patient's susceptibility to claustrophobia may also be taken into account when determining the fit score of a patient interface device. Different characteristics may be given different weights in determining the fit score of the patient interface device. Various algorithms have been developed, and continue to be developed, for determining the fit score of a patient interface for a patient, and, for purposes of the disclosed concept, any of these algorithms may be employed in determining the fit score of a patient interface device for a patient.

Generally, the patient interface device having the highest fit score will be recommended to a patient. The patient will generally continue to use the patient interface device until it is worn out or the patient no longer needs the patient interface device. After receiving the patient interface device, the patient will be unaware of patient interface devices that may be more suitable for them (e.g., having a higher fit score than their current patient interface device). For example, new patient interface devices may be released that are more suitable for the patient, but the patient will be unaware of these patient interface devices.

In an embodiment of the disclosed concept, when a new patient interface device is released, a fit score for the new patient interface will be determined. If the fit score for the new patient interface device is better than the fit score for the patient's current patient interface device, a notification will be pushed to the patient to notify them of the new patient interface device. In this manner, a patient can be notified of the new patient interface device that is more suitable for them and choose to use the new patient interface device rather than their current patient interface device.

While embodiments of the disclosed concept are described with respect to determining the suitability of a patient interface device for a patient, the disclosed concept is not limited thereto. The disclosed concept may be employed in conjunction with determining the suitability of various types of products for consumers. For example, the disclosed concept may be employed to notifying a consumer of any newly released product that is more suitable to the consumer than the consumer's current product.

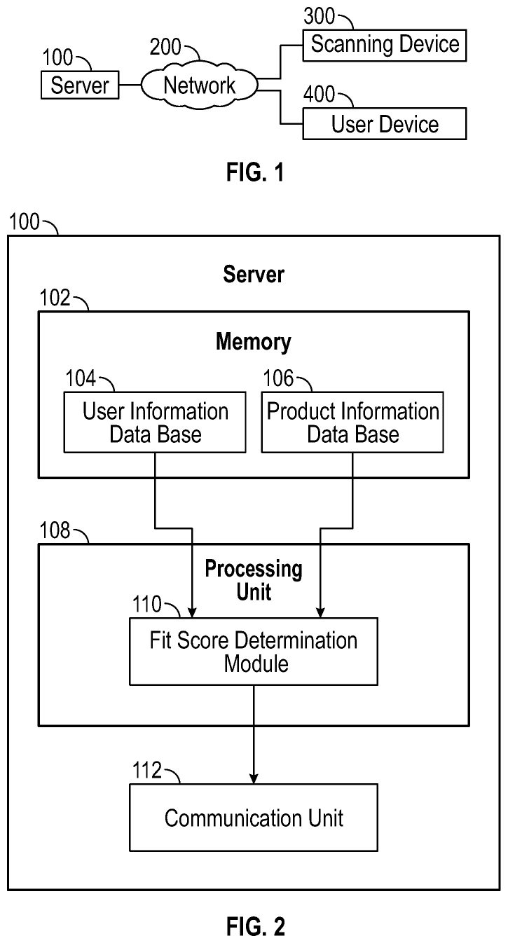

FIG. 1 is a schematic diagram of a patient interface selection system in accordance with an example embodiment of the disclosed concept. The patient interface selection system includes a server 100, a scanning device 300, and a user device 400 that are connected to a network 200. Server 100, scanning device 300, and user device 400 may communicate with each other via network 200. In an embodiment, network 200 may be the Internet. However, it will be appreciated that other types of communication networks may be employed without departing from the scope of the disclosed concept.

Scanning device 300 is structured to capture the geometry of the patient and communicate the geometry of the patient to server 100. Scanning device 300 may be, for example, a 3D scanner structured to perform a 3D scan of the patient's face and communicate the 3D scan to server 100. As another example, scanning device 300 may be a camera structured to capture one or more images of the patient's face and communicate the captured images to server 100. In another example, the captured images may be compiled into a 3D scan of the patient and communicated to server 100.

User device 400 may be an electronic device such as a computer, tablet, mobile phone or other similar devices. User device 400 may be accessible by the patient or others associated with the patient such as an equipment manufacturer or a medical provider. User device 400 is structured to receive notifications from server 100.

Server 100 may be one or multiple distributed servers connected to network 200. Sever 100 is structured to receive information, such as geometry of the patient, from scanning device 300 or other devices. Server 100 is also structured to receive and store information such as the geometry of the patient or other information associated with the patient such as characteristics of the patient and the fit score of the patient's current patient interface device. Server 100 is also structured to receive and store information associated with one or more patient interface devices. The information associated with the one or more patient interface devices may be, without limitation, geometry of the one or more patient interface devices. Server 100 is also structured to use the information associated with the patient and the information associated with one or more patient interface devices to determine if the one or more patient interface devices has a fit score that is higher than the fit score for the patient's current patient interface device. If the fit score of one of the patient interface devices is higher than the fit score of the patient's current patient interface device, server 100 is structured to push a notification to user device 400 to notify the patient that a patient interface device with a higher fit score is available. The notification may include identification information of the patient interface device with the higher fit score. The notification may also include a degree of improvement the patient interface device with the higher fit score provides over the patient's current patient interface device.

FIG. 2 is a schematic diagram of server 100 in accordance with an example embodiment of the disclosed concept. In an example embodiment, server 100 includes a memory 102 and a processing unit 108. Processing unit 108 may include a processor that may be, for example and without limitation, a microprocessor, a microcontroller, or some other suitable processing device or circuitry, that interfaces with the memory. Memory 102 can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory.

Memory 102, in an example embodiment, includes a user information database 104 and a product information database 106. User information database 104 is structured to store information associated with the patient. The information associated with the patient may include, among other things, a geometry of the patient and/or patient's face, characteristics of the patient, and information about the patient's current patient interface device along with a fit score for the patient interface device. Product information database 106 is structured to store information associated with one or more patient interface devices. The information associated with the one or more patient interface devices may include identification information of the one or more patient interface devices and geometries of the one or more patient interface devices.

Processing unit 108 includes a fit score determination module 110. Fit score determination module 110 is structured to use the information associated with the patient and the information associated with the one or more patient interface devices to determine the fit scores of the one or more patient interface devices. Any suitable method may be used for determining the fit scores of the patient interface devices without departing from the scope of the disclosed concept. Fit score determination module 110 is structured to determine if the determined fit scores of one or more of the patient interface devices is higher than the fit score of the patient's current patient interface device.

Server 100 also include a communication unit 112 structured to communicate with user device 400 (e.g., via network 200). If fit score determination module 110 determines that the fit scores of one or more of the patient interface devices is higher than the fit score of the patient's current patient interface device, fit score determination module 110 is structured to control communication unit 112 to push a notification to user device 400 to indicate that the one or more patient interface devices are available that are more suitable than the patient's current patient interface device.

In an example embodiment, the notification may also include an indication of how much better the patient interface device with the best fit score would fit the patient. For example, the notification may indicate that the patient interface device with the best fit score may benefit the patient by, for example, 20%, or may provide, for example, 25% more comfort.

In an example embodiment, fit score determination module 110 is structured to monitor when a new patient interface device is available (e.g., when information on the new patient interface device is added to product information database) and responsively determine the fit score corresponding to the new patient interface device and push the notification to the patient if the fit score for the new patient interface device is better than the fit score for the patient's current patient interface device. In this manner, the patient can be notified if a new patient interface device would be more suitable than the patient's current patient interface device without further input from the patient.

Also, in an example embodiment, fit score determination module 110 is structured to periodically determine fit scores corresponding to one or more patient interface devices whose information is stored in product information database 106 and to push a notification to the patient if one of the determined fit scores is better than the fit score corresponding to the patient's current patient interface device. In this manner, over time, the patient can be notified if any recently released patient interface device would be more suitable than the patient's current patient interface device without further input from the patient.

It will be appreciated that while the examples of FIGS. 1 and 2 are described with respect to patients and patient interface devices, the devices and systems may be employed to various consumers and types of products without departing from the scope of the disclosed concept.

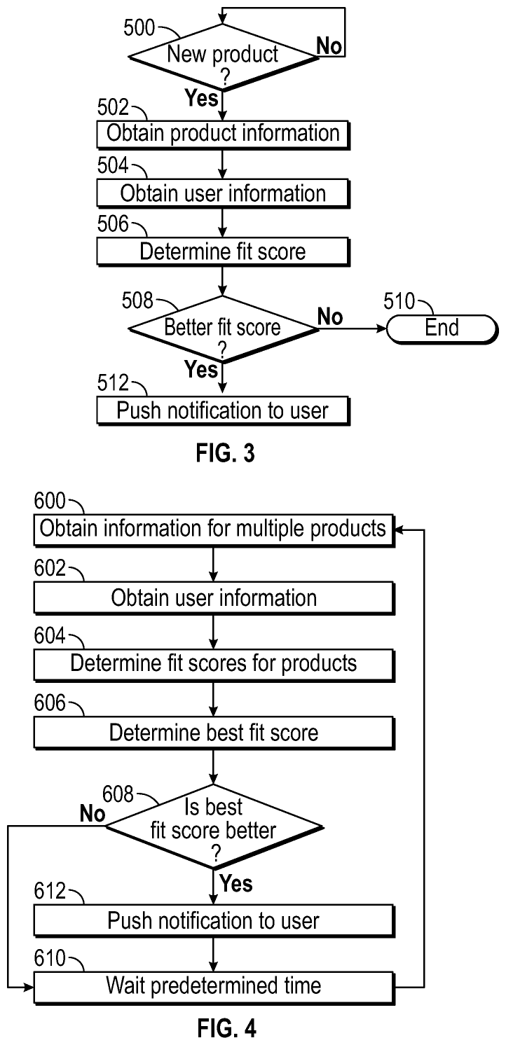

FIG. 3 is a flowchart of a method of assisting with patient interface device selection in accordance with an example embodiment of the disclosed concept. The method may be implemented, for example, with server 100. However, it will be appreciated that the method may be implemented in other devices without departing from the scope of the disclosed concept.

The method begins at 500 where server 100 determines if a new patient interface device is available. The method remains at 500 until it is determined that a new patient interface device is available, and then the method proceeds to 502. At 502, information on the new patient interface device is obtained, for example, from product information database 106. The patient interface device information may include, for example, a geometry of the patient interface device. At 504, information on a patient is obtained, for example, from user information database 104. The information on the patient may include, for example, a geometry of the patient's face, a fit score corresponding to the patient's current patient interface device, and other characteristics of the patient.

The method then proceeds to 506 where a fit score for the new patient interface device is determined with, for example, fit score determination module 110. The fit score may be determined using any suitable method. For example, the geometry of the new patient interface device and the geometry of the patient's face may be compared as part of determining the fit score. However, it will be appreciated that other information, such as the patient's characteristics, may be taken into account in determining the fit score of the new patient interface device. Once the fit score of the new patient interface device is determined, the method proceeds to 508. At 508, it is determined whether the fit score corresponding to the new patient interface device is better than the fit score corresponding to the patient's current patient interface device. If it is not, the method ends at 510. However, if the fit score corresponding to the new patient interface device is better than the fit score corresponding to the patient's current patient interface device, the method proceeds to 512. At 512, a notification is pushed to the patient indicating that the new patient interface device is more suitable for the patient. For example, server 100 may push the notification to user device 400. In an example embodiment, the notification may also include an indication of how much better the new patient interface device would fit the patient. For example, the notification may indicate that the new patient interface device may benefit the patient by, for example, 20%, or may provide, for example, 25% more comfort over the patient's current patient interface device.

It will be appreciated that while the method of FIG. 3 is described with respect to patients and patient interface devices, the method may be employed to various consumers and types of products without departing from the scope of the disclosed concept.

FIG. 4 is a flowchart of a method of assisting with patient interface device selection in accordance with another example embodiment of the disclosed concept. The method may be implemented, for example, with server 100. However, it will be appreciated that the method may be implemented in other devices without departing from the scope of the disclosed concept. The method of FIG. 4 has some similarity with the method of FIG. 3, except that the method of FIG. 4 relates to periodically determining fit scores corresponding to patient interface devices, as will be described herein.

The method begins at 600 where information is obtained for multiple patient interface devices, for example, from product information database 106. It will be appreciated that at 600, information may be obtained for a single patient interface device without departing from the scope of the disclosed concept. The information on the patient interface devices may include, for example, geometries of the patient interface devices. At 602, information of a patient is obtained, for example, from user information database 104. The patient information may include information on the patient may include, for example, a geometry of the patient's face, a fit score corresponding to the patient's current patient interface device, and other characteristics of the patient.

At 604, fit scores fit scores corresponding to patient interface devices are determined with, for example, fit score determination module 110. The fit scores may be determined using any suitable method. For example, the geometries of the patient interface devices and the geometry of the patient's face may be compared as part of determining the fit score. However, it will be appreciated that other information, such as the patient's characteristics, may be taken into account in determining the fit score of the new patient interface device. Once the fit scores of the new patient interface devices are determined, the method proceeds to 608. At 608, it is determined whether the best fit score among those determined for the patient interface devices is better than the fit score corresponding to the patient's current patient interface device. If it is not, the method proceeds to 610. At 610, a predetermined time is waited before the method proceeds back to 600. The predetermined time may be any suitable amount of time. For example, the predetermined time may be a day, a week, or some other suitable predetermined time.

If the best fit score for the patient interface devices is better than the fit score corresponding to the patient's current patient interface device, the method proceeds to 612. At 612, a notification is pushed to the patient indicating that the patient interface device having the best fit score is more suitable for the patient. For example, server 100 may push the notification to user device 400. In an example embodiment, the notification may also include an indication of a degree of improvement of the patient interface device with the best fit score over the patient's current patient interface device. For example, the notification may indicate that the patient interface device with the best fit score may benefit the patient by, for example, 20%, or may provide, for example, 25% more comfort over the patient's current patient interface device.

With the method of FIG. 4, fit scores for patient interface devices can be periodically determined and the patient can be notified if a patient interface device has a better fit score than the patient's current patient interface device. In this manner, fit scores for recently released patient interface devices can be compared to the fit score for the patient's current patient interface device and the patient can be notified of a patient interface device with a better fit score. Thus, without further input on the patient's part, the patient can be made aware of a better fitting patient interface device that the patient may otherwise not been aware of.

It will be appreciated that while the method of FIG. 4 is described with respect to patients and patient interface devices, the method may be employed to various consumers and types of products without departing from the scope of the disclosed concept.

Although the disclosed has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present disclosed concept contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system comprising:
a user information database structured to store first information corresponding to a consumer and a first fit score corresponding to a current product associated with the consumer;
a product information database structured to store second information corresponding to a new product;
a communication unit structured to communicate with a user device; and
a processing unit including a fit score determination module structured to determine a second fit score of the new product based on the first information and the second information, to determine if the second fit score is greater than the first fit score, and to control the communication unit to push a notification to the user device in response to determining that the second fit score is greater than the first fit score.

2. The system of claim 1, wherein the current product is a first patient interface device, the new product is a second patient interface device, and the consumer is a patient.

3. The system of claim 1, wherein the first information includes one or more characteristics of the consumer, and wherein the one or more characteristics of the consumer includes a geometry of the consumer.

4. The system of claim 3, wherein the second information includes one or more characteristics of the new product, wherein the one or more characteristics of the new product includes a geometry of the new product.

5. The system of claim 4, wherein the fit score determination module is structured to determine the second fit score based on a comparison of the geometry of the consumer and the geometry of the new product.

6. The system of claim 1, wherein the fit score determination unit is structured to determine when the second information corresponding to the new product is stored in the product database and to responsively determine the second fit score of the new product based on the first information and the second information, determine if the second fit score is greater than the first fit score, and control the communication unit to push the notification to the user device in response to determining that the second fit score is greater than the first fit score.

7. The system of claim 1, wherein the fit score determination module structured to periodically determine the second fit score of the new product based on the first information and the second information, to determine if the second fit score is greater than the first fit score, and to control the communication unit to push the notification to the user device in response to determining that the second fit score is greater than the first fit score.

8. The system of claim 1, wherein the product database structured to store a plurality of second information corresponding to a plurality of new products, and wherein the fit score determination module structured to determine the second fit score of multiple of the plurality of new products based on the first information and the plurality of second information, to determine if any of the second fit scores is greater than the first fit score, and to control the communication unit to push the notification to the user device in response to determining that any of the second fit scores is greater than the first fit score

9. The system of claim 1, wherein the notification includes information on an identity of the new product.

10. The system of claim 1, wherein the fit score determination module is structured to determine a degree of improvement of the new product over the current product, and wherein the notification includes information on the degree of improvement.

11. A method of assisting with product selection, the method comprising:
obtaining first information corresponding to a consumer and a first fit score corresponding to a current product associated with the consumer;
obtaining second information corresponding to a new product;
determining a second fit score of the new product based on the first information and the second information;
determining if the second fit score is greater than the first fit score; and
in response to determining that the second fit score is greater than the first fit score, pushing a notification to a user device.

12. The method of claim 11, wherein the current product is a first patient interface device, the new product is a second patient interface device, and the consumer is a patient.

13. The method of claim 11, further comprising:
determining if the new product is available, and
wherein obtaining second information corresponding to the new product include obtaining second information corresponding to the new product in response to determining that the new product is available.

14. The method of claim 11, further comprising:
waiting a predetermined period of time after determining if the second fit score is greater than the first fit score; and
after waiting the predetermined period of time:
obtaining first information corresponding to the consumer and the first fit score corresponding to the current product associated with the consumer;
obtaining third information corresponding to a second new product;
determining a third fit score of the second new product based on the first information and the third information;
determining if the third fit score is greater than the first fit score; and
in response to determining that the third fit score is greater than the first fit score, pushing a second notification to the user device.

15. The method of claim 11, further comprising: determining a degree of improvement of the new product over the current product, wherein the notification includes information on the degree of improvement.
